(19) United States
(12) Patent Application Publication
(10) Pub. No.: US 2023/0248571 A1
(43) Pub. Date: Aug. 10, 2023

(54) Systems & Methods for Combined Femto-Phaco Cataract Surgery

(71) Applicant: Lensar, Inc., Orlando, FL (US)

(72) Inventor: Douglas Patton, Irvine, CA (US)

(73) Assignee: Lensar, Inc., Orlando, FL (US)

(21) Appl. No.: 18/092,379

(22) Filed: Jan. 2, 2023

Related U.S. Application Data
(63) Continuation of application No. 16/219,700, filed on Dec. 13, 2018, now Pat. No. 11,540,943.
Application No. 16/219,700 is a continuation of application No. 14/666,908, filed on Mar. 24, 2015, now Pat. No. 10,285,858.
Application No. 14/666,908 is a continuation of application No. 13/648,196, filed on Oct. 9, 2012, now Pat. No. 8,986,290.
(60) Provisional application No. 61/543,930, filed on Oct. 6, 2011.

(51) Int. Cl. A61F 9/008; A61F 9/007

(52) U.S. Cl. CPC A61F 9/00802; A61F 9/00825; A61F 9/00745; A61F 2009/0087; A61F 2009/00887; A61F 2009/00872

(57) ABSTRACT

Devices to perform femtolaser ablation and phacoemulsification are physically and/or operationally combined. In some embodiments the femtolaser ablation and phacoemulsification are housed together, and in other embodiments they are housed separately, but operated through a common display screen. At least some software can be shared by the femtolaser ablation and phacoemulsification functionalities. A non-transitory computer-readable memory can provide data that can be used to operate each of at least one femtolaser ablation functionality and at least one phacoemulsification functionality.

This application claims priority to U.S. Ser. No. 14/666,908, filed Mar. 24, 2015, which claims priority to U.S. Ser. No. 13/648,196, filed Oct. 9, 2012, which claims priority to U.S. Provisional Application No. 61/543,930, filed Oct. 6, 2011, each of which is incorporated herein by reference in its entirety.

### FIELD OF THE INVENTION

The field of the invention is cataract surgery.

### BACKGROUND

Modern cataract surgery is commonly performed using phacoemulsification, which is colloquially known as “phaco”. In that process the internal lens of an eye is emulsified with an ultrasonic handpiece, and the detritus is aspirated from the eye. Aspirated fluids are replaced with irrigation of balanced salt solution. As with other cataract extraction procedures, an intraocular lens implant (IOL), is placed into the remaining lens capsule. See http://en.wikipedia.org/wiki/Phacoemulsification.

The referenced Wikipedia article, as well as all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

The phaco probe is an ultrasonic handpiece with a titanium or steel needle. The tip of the needle vibrates at ultrasonic frequency to sculpt and emulsify the cataract, while the pump aspirates particles through the tip. In some techniques, a second fine steel instrument called a “chopper” is used from a side port to help with chopping the nucleus into smaller pieces. The cataract is usually broken into two or four pieces and each piece is emulsified and aspirated out with suction. The nucleus emulsification makes it easier to aspirate the particles. After removing all hard central lens nucleus with phacoemulsification, the softer outer lens cortex is removed with suction only.

Phacoemulsification surgery involves the use of a machine with microprocessor-controlled fluid dynamics. Emulsification power applied through the probe, vacuum strength of the aspirator, and other operating parameters of the phaco device are controlled through software. Examples of graphic user interfaces of popular phacoemulsification software are depicted in prior art FIGS. 1 and 2.

Before phacoemulsification can be performed, one or more incisions are typically made in the eye to allow the introduction of surgical instruments. The surgeon then removes the anterior face of the capsule that contains the lens inside the eye. During that part of the procedure, difficulties sometimes arise during the incision portion of the procedure, potentially causing damage to the eye.

It is known to use femtosecond laser pulses to make incisions into the eye, and recently devices are available for remediating cataracts using femtolaser ablation. One device currently in the marketplace for accomplishing that is Technolas Perfect Vision's™ VICTUS™ Femtosecond Laser Platform. See http://www.technolaspv.com/dasat/index.php?cid=100858. Femtolaser ablation of cataracts can remove even fairly large cataracts quite quickly, but (1) some portions of cataract can remain, requiring phacoemulsification, and (2) detritus must still be removed using irrigation and aspiration.

Currently, cataract remediation using femtolaser ablation followed by phacoemulsification is performed by two entirely separate machines, requiring translocation and re-setup of the patient. This increases the time required for the procedure, and can cause undue stress on the patient.

Thus, there is a need to combine femtolaser ablation and phacoemulsification either in the same device, or at least in separate devices that are operated using a combined software platform.

### SUMMARY OF THE INVENTION

The inventive subject matter provides apparatus, systems and methods in which devices to perform femtolaser ablation and phacoemulsification are physically and/or operationally combined.

In one aspect, at least one subsystem of the femtolaser ablation functionality is housed in a common housing with at least one subsystem of the phacoemulsification functionality.

In another aspect, a display screen displays both a first graphical user interface for the femtolaser ablation functionality and a second graphical user interface for the phacoemulsification functionality.

In another aspect, software includes instructions disposed on a non-transitory computer-readable medium that are common to operation of both the femtolaser ablation functionality and the phacoemulsification functionality.

In another aspect a non-transitory computer-readable memory provides data that can be used to operate each of the femtolaser ablation functionality and the phacoemulsification functionality.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a screen shot of a prior art graphical user interface for a phacoemulsification device.

FIG. 2 is a screen shot of an alternative prior art graphical user interface for a phacoemulsification device.

FIG. 3 is a prior art representation of the phacoemulsification device of FIGS. 1 and 2.

FIG. 4 is a prior art representation of a display of the phacoemulsification device of FIGS. 1 and 2, showing an eye during phacoemulsification.

FIG. 5 is a prior art representation of a display of the phacoemulsification device of FIGS. 1 and 2, showing an eye during aspiration.

FIG. 6 is a prior art screen shot of a prior art graphical user interface for a femtolaser ablation device.

FIG. 7 is a screen shot of another prior art graphical user interface of the femtolaser ablation device of FIG. 3.

FIG. 8 is a prior art representation medical personnel using the femtolaser ablation device of FIGS. 6 and 7.

FIG. 9 is a prior art representation medical personnel using the femtolaser ablation device of FIGS. 6 and 7.

FIG. 10 is another prior art representation of a femtolaser ablation device.

FIG. 11 is a schematic of a graphical user interface of software configured to control both a femtolaser ablation device and a phacoemulsification device.

FIG. 12 is a schematic of the graphical user interface of FIG. 11, showing a menu of procedures that can be selected for the femtolaser ablation device.

FIG. 13 is a schematic of the graphical user interface of FIG. 11, showing a menu of procedures that can be selected for the phacoemulsification device.

FIG. 14 is a schematic of the graphical user interface of FIG. 11, concurrently showing menus of procedures that can be selected for the femtolaser ablation device and the phacoemulsification device

FIG. 15 is a schematic of a graphical user interface concurrently showing menus of procedures that can be selected for a device that combines femtolaser ablation functionality and phacoemulsification functionality.

FIG. 16 is a schematic of a device that combines femtolaser ablation functionality and phacoemulsification functionality, using a common GUI control platform.

FIG. 17 is a schematic of a device that utilizes a common GUI control platform to control physically separate femtolaser ablation and phacoemulsification devices.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

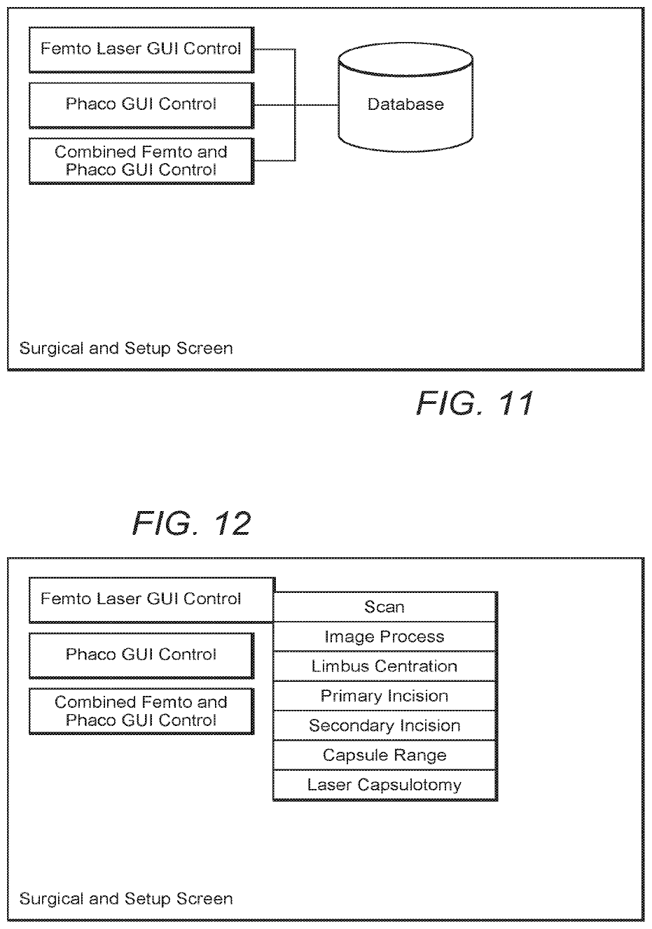

As seen in FIGS. 11-15, it is contemplated that a single GUI and/or software can combine control of ophthalmic phacoemulsification and femtolaser functionalities for cataract removal. These can be combined in many diverse ways for surgical use on at least one of a computer touch screen, a set of dedicated buttons, a foot pedal, and a voice interface.

FIG. 11 is a schematic of a graphical user interface of software configured to control both a femtolaser ablation device and a phacoemulsification device. The platform can be local to one or more of the devices, and can alternatively or additionally be at least partially disposed on one or more distal servers (e.g., cloud, PaaS, IaaS, SaaS, etc.). Each of the devices shown in the figures should be interpreted as including electronics and software needed to operate the device, and the software should be interpreted as capable of reading and utilizing data from one or more databases.

Devices shown in the figures with connecting lines should be interpreted as being at least functionally coupled to one another, and in some embodiments physically coupled to one another. Thus, a display device shown with a connecting line to a femtolaser ablation device should be interpreted as being at least electronically coupled to one another. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. The terms “coupled to” and “coupled with” are also use euphemistically to mean “communicatively coupled with” in a networking sense where at least two networked elements are able to communicate with each other over a network possibly via one or more intermediary devices.

It should be noted that any language directed to a computer or software platform should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

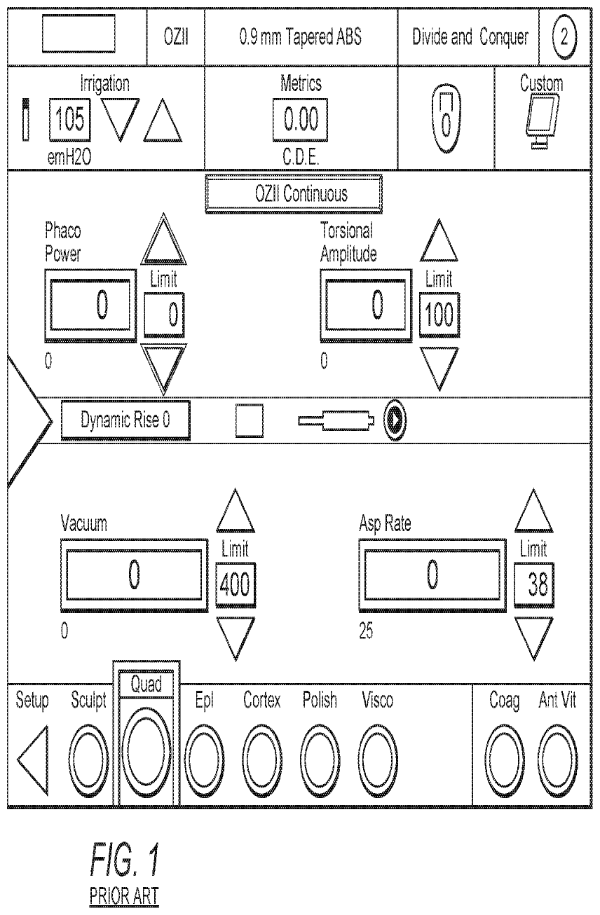

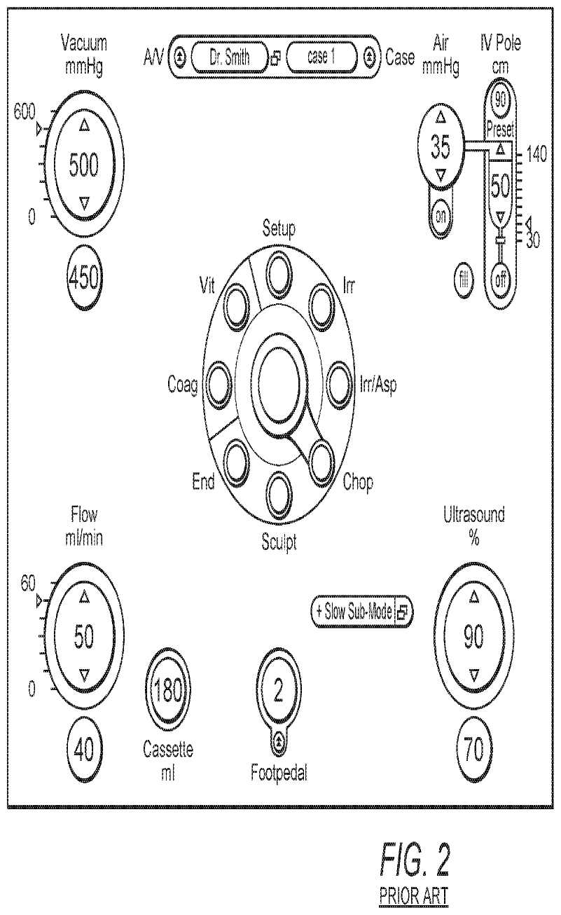

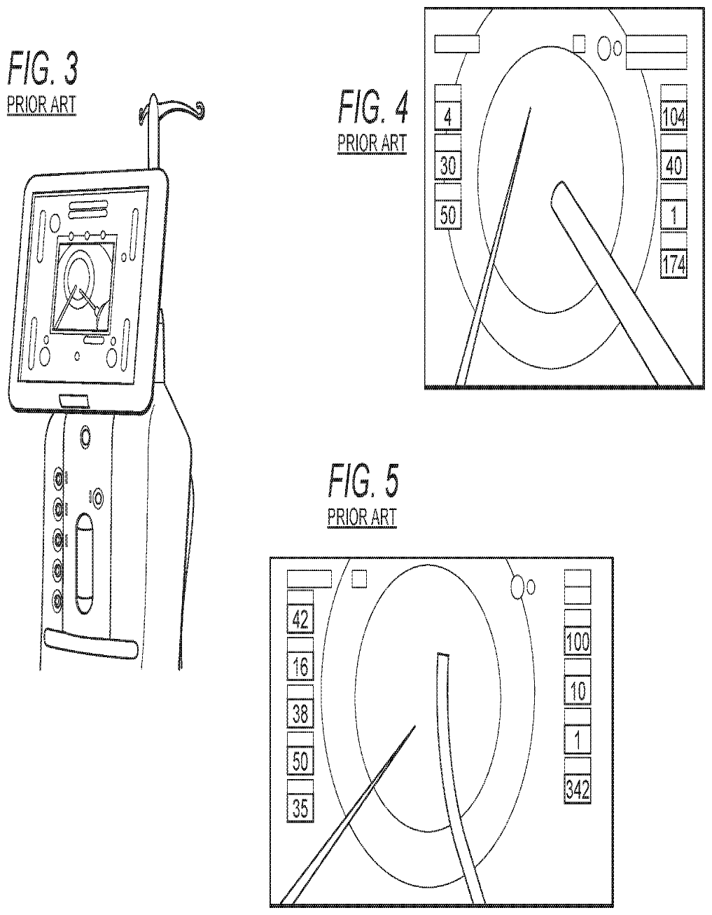

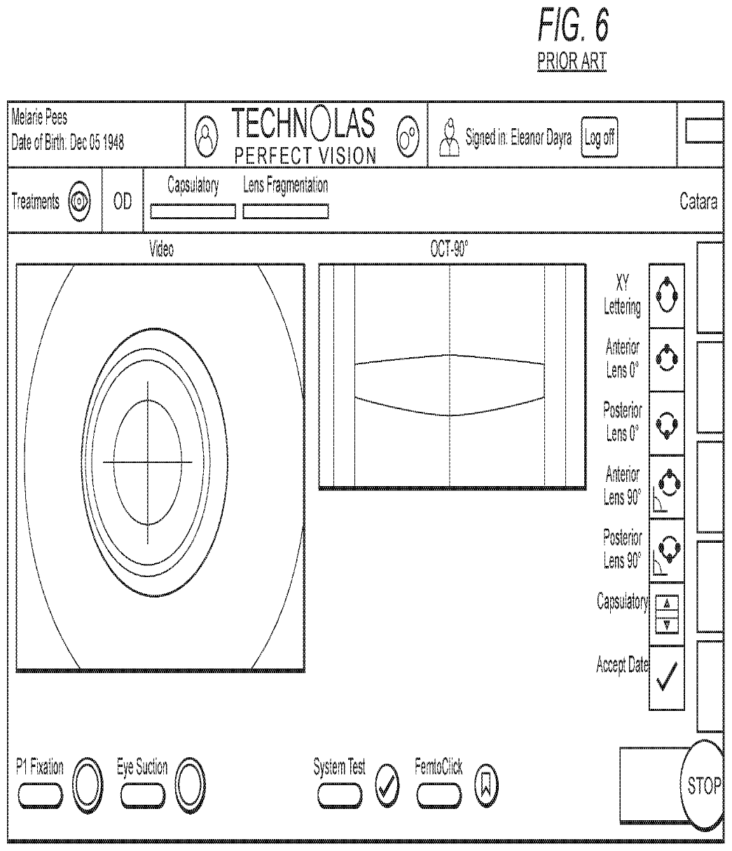

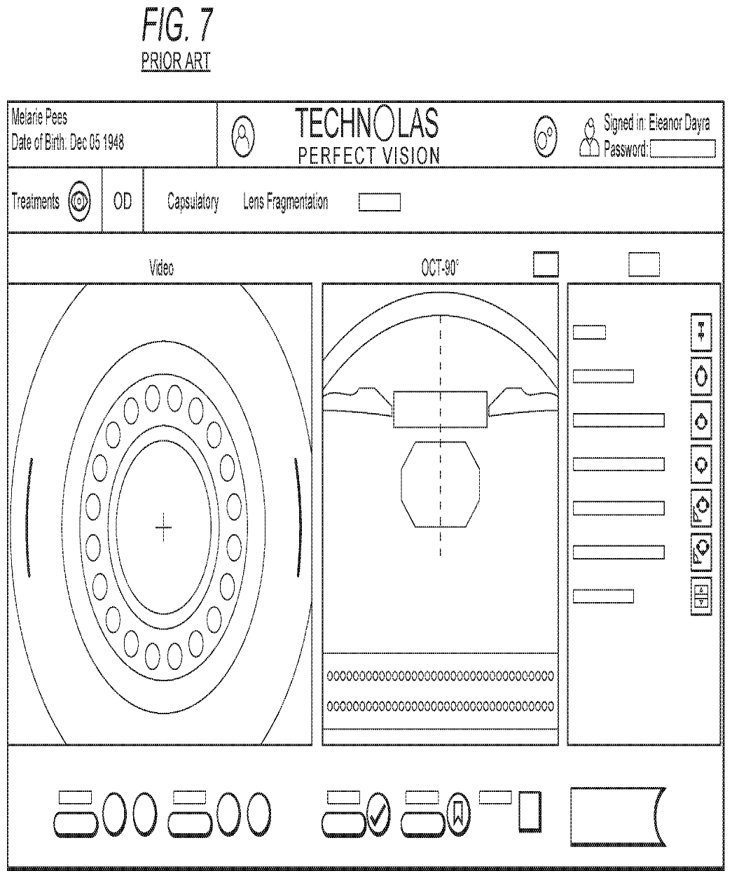

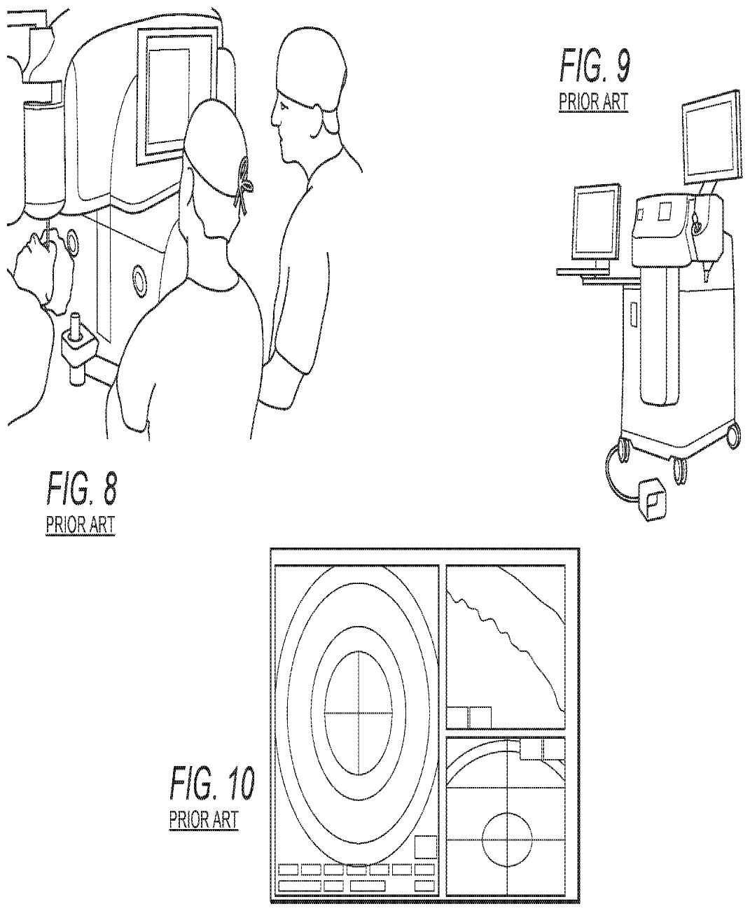

FIG. 12 is a schematic of the graphical user interface of FIG. 11, showing a menu of procedures that can be selected for the femtolaser ablation device. These are exemplary procedures only, and contemplated femtolaser GUI interfaces may list other or additional procedures. One should also appreciate that each of FIGS. 12-15 are merely schematics, and commercial embodiments of the interfaces would be much more detailed and user friendly, such as along the lines of that shown in FIG. 1 or 2.

FIG. 13 is a schematic of the graphical user interface of FIG. 11, showing a menu of procedures that can be selected for the phacoemulsification device.

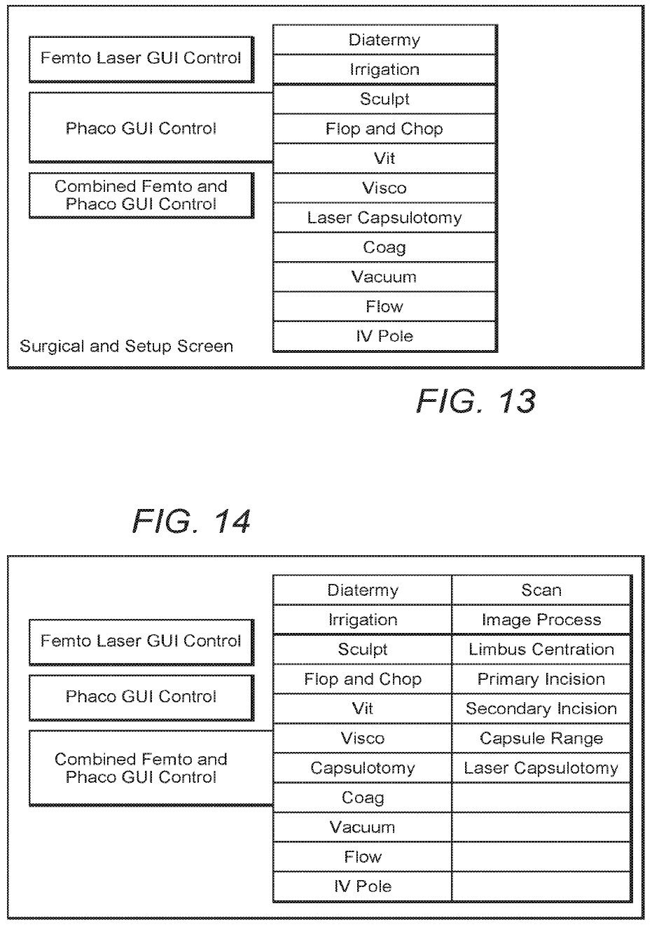

FIG. 14 is a schematic of the graphical user interface of FIG. 11, concurrently showing menus of procedures that can be selected for separate femtolaser ablation and phacoemulsification devices.

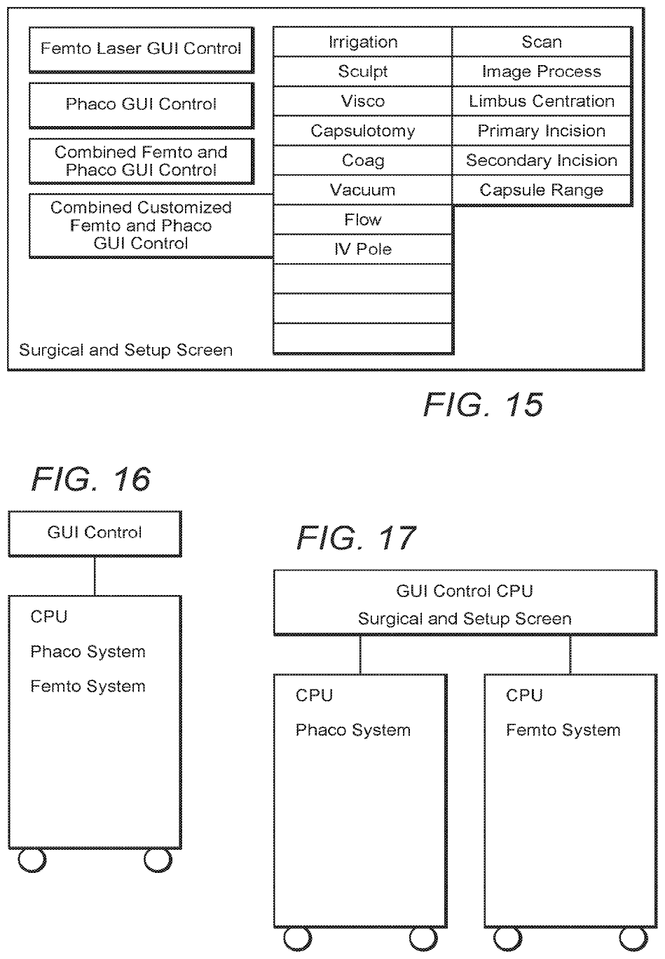

FIG. 15 is a schematic of a graphical user interface concurrently showing menus of procedures that can be selected for a device that combines femtolaser ablation functionality and phacoemulsification functionality. This interface differs from that shown in FIG. 14 because the listing of functionalities is customized in some manner, for example by including custom functionalities not programmed into off-the-shelf devices, and/or by hiding functionalities that might otherwise be available. Such customization of main menu and/or submenu procedures for phaco and femto could be arranged by the manufacturer and/or or surgeon in an order that is thought to best fit the process of cataract removal and soft lens insertion for a particular practitioner or office.

FIG. 16 is a schematic of a device that combines femtolaser ablation functionality and phacoemulsification functionality, using a common GUI control platform. FIG. 17 is a schematic of a device that utilizes a common GUI control platform to control physically separate femtolaser ablation and phacoemulsification devices. Each of the interfaces of FIGS. 12-15 could be used with each of the embodiments shown in FIGS. 16 and 17.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1-4. (canceled)

5. A system for performing laser and ultrasonic procedures on the eye, the system comprising:
a) a combined device having a laser device and a phacoemulsification device;
b) a control platform configured to provide submenus for both a phacoemulsification procedure and a laser procedure on a GUI; wherein the GUI is associated with the combined device; and,
c) at least one of the GUI, a computer touchscreen, a dedicated button, a foot pedal, and voice interaction configured to combine in multiple different ways, the phacoemulsification and laser procedures provided by the submenus.

6. The system of claim 5, wherein the laser device comprises a pulsed laser.

7. The system of claim 5, wherein the laser device comprises a femto second laser.

8. The system of claim 5, wherein the phacoemulsification device comprises a handpiece configured to vibrate a tip at an ultrasonic frequency.

9. The system of claims 6, wherein the phacoemulsification device comprises a handpiece configured to vibrate a tip at an ultrasonic frequency.

10. The system of claims 7, wherein the phacoemulsification device comprises a handpiece configured to vibrate a tip at an ultrasonic frequency.

11. The system of claim 5, wherein at least a portion of the laser device is housed in a common housing with at least a portion of the phacoemulsification device.

12. The system of claim 6, wherein at least a portion of the laser device is housed in a common housing with at least a portion of the phacoemulsification device.

13. The system of claim 9, wherein at least a portion of the laser device is housed in a common housing with at least a portion of the phacoemulsification device.

14. The system of claim 10, wherein at least a portion of the laser device is housed in a common housing with at least a portion of the phacoemulsification device.

15. The system of claim 5, wherein the laser procedure includes one or more of scan, image process, limbus centration, primary incision and secondary incision.

16. The system of claim 6, wherein the laser procedure includes one or more of capsule range and laser capsulotomy.

17. The system of claim 7, comprising a database having data that describes a main menu and the submenus for both the phacoemulsification and the laser procedures.

18. A method of operating a combined device having a laser and a phacoemulsification device, the method comprising:
a) providing submenus for both a phacoemulsification procedure and a laser procedure on a GUI; wherein the GUI is associated with a combined device comprising a laser and a phacoemulsification device; and,
b) using at least one of the GUI, a computer touchscreen, a dedicated button, a foot pedal, and voice interaction to combine in multiple different ways, the phacoemulsification and the laser procedures provided by the submenus.

19. The method of claim 18, wherein the laser is a pulsed laser.

20. The method of claim 18, wherein the laser is a femto second laser.

21. The method of claim 18, wherein the phacoemulsification procedure comprises diathermy.

22. The method of claim 18, wherein the phacoemulsification procedure comprises irrigation

23. The method of claim 18, wherein the phacoemulsification procedure comprises sculpt.

24. The method of claim 18, wherein the phacoemulsification procedure comprises chop.

25. The method of claim 18, wherein the phacoemulsification procedure comprises vit.

26. The method of claim 18, wherein the phacoemulsification procedure comprises visco.

27. The method of claim 18, wherein the laser procedure comprises one or more of scan, image process, limbus centration, primary incision and secondary incision.

28. The method of claim 19, wherein the laser procedure comprises capsule range

29. The method of claim 20, wherein the laser procedure comprises laser capsulotomy.

30. The method of any claim 20, comprising obtaining data from a database having the data, wherein the data describes a main menu and the submenus for both the phacoemulsification and the femto laser procedures.

31. The method of claim 20, wherein the laser procedure comprises one or more of scan, image process, limbus centration, primary incision and secondary incision; and, comprising obtaining data from a database having the data, wherein the data describes a main menu and the submenus for both the phacoemulsification and the femto laser procedures.

32. The method of claim 18, wherein the laser procedure comprises capsule range; and comprising obtaining data from a database having the data, wherein the data describes a main menu and the submenus for both the phacoemulsification and the femto laser procedures.

33. The method of claim 18, wherein the laser procedure comprises laser capsulotomy; and comprising obtaining data from a database having the data, wherein the data describes a main menu and the submenus for both the phacoemulsification and the femto laser procedures.

34. A system for performing laser and ultrasonic procedures on the eye for the treatment of cataracts, the system comprising:
a) a combined device having a laser device and a phacoemulsification device;
b) a control platform configured to provide submenus for both a phacoemulsification procedure and a laser procedure on a GUI; wherein the GUI is associated with the combined device; and,
c) at least one of the GUI and a computer touchscreen configured to combine in multiple different ways, the phacoemulsification and laser procedures provided by the submenus.

35. A system for performing laser and ultrasonic procedures on the eye for the treatment of cataracts, the system comprising:
a) a combined device having a laser device and a phacoemulsification device;
b) a control platform configured to provide submenus for both a phacoemulsification procedure and a laser procedure on a GUI; wherein the GUI is associated with the combined device; and,
c) at least one of the GUI and a foot pedal configured to combine in multiple different ways, the phacoemulsification and laser procedures provided by the submenus.

36. A system for performing laser and ultrasonic procedures on the eye for the treatment of cataracts, the system comprising:
a) a combined device having a laser device and a phacoemulsification device;
b) a control platform configured to provide submenus for both a phacoemulsification procedure and a laser procedure on a GUI; wherein the GUI is associated with the combined device; and,
c) at least one of the GUI and a dedicated button configured to combine in multiple different ways, the phacoemulsification and laser procedures provided by the submenus.

37. The system of claim 34, 35 or 36, comprising a database having data that describes a main menu and the submenus for both the phacoemulsification and the laser procedures.

38. The system of claim 34, 35 or 36, wherein at least a portion of the laser device is housed in a common housing with at least a portion of the phacoemulsification device.

39. The system of claim 34, 35 or 36, wherein at least a portion of the laser device is housed in a common housing with at least a portion of the phacoemulsification device; and, comprising a database having data that describes a main menu and the submenus for both the phacoemulsification and the laser procedures.
